(19) United States
(12) Patent Application Publication
(10) Pub. No.: US 2005/0138692 A1
(43) Pub. Date: Jun. 23, 2005

(54) Production of cancer-specific antibodies in plants

(75) Inventors: Hilary Koprowski, Wynnewood, PA (US); Kisung Ko, Drexel Hill, PA (US); Pauline Rudd, Oxford (GB); Yoram Tekoah, Omer (IL); Raymond Dwek, Oxford (GB)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignees: Biotechnology Foundation, Inc., Philadelphia, PA (US); The Chancellor, Masters and Scholars of the University of Oxford, University of Oxford, Oxford (GB)

(21) Appl. No.: 10/860,322

(22) Filed: Jun. 2, 2004

Related U.S. Application Data
(60) Provisional application No. 60/475,311, filed on Jun. 2, 2003.

(51) Int. Cl. A01H 1/00; C12N 15/82; C12P 21/04; C07K 16/18; C12N 5/04

(52) U.S. Cl. 800/288; 435/468; 435/419; 530/387.1

(57) ABSTRACT

The invention as described herein provides compositions and methods for cancer immunotherapy and cancer detection. In particular, the invention discloses plant-derived human monoclonal antibodies that bind human carcinoma antigens in cancer cell lines.

### I. FIELD OF THE INVENTION

The invention is directed to immunological compositions and methods of making and using same. In particular, the invention is directed to plant-derived antibodies and their use as immunotherapeutic agents against human cancer.

### II. BACKGROUND OF THE INVENTION

Since the expression of functional monoclonal antibodies in transgenic plants was described by Hiatt et al. (1989) Nature 342:76-79, transgenic plants have been considered as an efficient production system for functional therapeutic monoclonal antibody (Ma et al. (1998) Nature Medicine 4:601-606). Monoclonal antibodies isolated from plant tissues have advantages such as lack of animal pathogenic contaminants, relatively inexpensive plant cultivation, and low cost of scale up for agricultural production compared to the conventional fermentation methods. Verch et al. (1998) J. Immunol. Methods 220:69-75 recently has reported that a full-length MAb CO17-1A was expressed and assembled using the tobacco mosaic virus (TMV) vector expression system in tobacco plant, but, there was no report on the binding activity of the MAb CO17-1A to colorectal carcinoma cells expressing Ag GA733-2.

The plant virus expression system is potentially more rapid and efficient than the establishment of transgenic plants. However, the system has several drawbacks. For example, plant virus expression systems require a virus transcript inoculation due to temporary, transient gene expression. Additionally, plant virus expression systems often display a very high mutation and deletion rate of foreign genes during plant RNA virus replication (Smith et al. (1997) Reprod. Fertil. Dev. 9:85-89). In contrast, plant non-viral expression systems have several advantages over the plant virus expression system, such as stable gene insertion and easy multiplication of transgenic plants through in vitro tissue culture or seedling (Koprowski et al. (2001) Vaccine 19:2735-2741).

Monoclonal antibody (MAb) technology has greatly impacted current thinking about cancer therapy and diagnosis. The elegant application of cell to cell fusion for the production of MAbs by Kohler and Milstein (Nature (London) 256:495 (1975)) spawned a revolution in biology equal in impact to that of recombinant DNA cloning. MAbs produced from hybridomas are already widely used in clinical studies and basic research, testing their efficacy in the treatment of human diseases including cancer, viral and microbial infections, and other diseases and disorders of the immune system.

Although they display exquisite specificity and can influence the progression of human disease, mouse MAbs, by their very nature, have limitations in their applicability to human medicine. Most obviously, since they are derived from mouse cells, they are recognized as foreign protein when introduced into humans and elicit immune responses. Similarly, since they are distinguished from human proteins, they are cleared rapidly from circulation.

Technology to develop MAbs that could circumvent these particular problems has met with a number of obstacles. This is especially true for MAbs directed to human tumor antigens, developed for the diagnosis and treatment of cancer. Since many tumor antigens are not recognized as foreign by the human immune system, they probably lack immunogenicity in man. In contrast, those human tumor antigens that are immunogenic in mice can be used to induce mouse MAbs which, in addition to specificity, may also have therapeutic utility in humans. In addition, most human MAbs obtained in vitro are of the IgM class or isotype. To obtain human MAbs of the IgG isotype, it has been necessary to use complex techniques (i.e., cell sorting) to first identify and isolate those few cells producing IgG antibodies. A need therefore exists for an efficient way to switch antibody classes at will for any given antibody of a predetermined or desired antigenic specificity.

Differences in post-translational modifications, such as glycosylation, have been shown to influence the properties of plant-derived proteins (Daniell et al., supra; Conrad et al. (1998) Plant Mol. Biol. 38:101-109; Mann et al. (2003) Nat. Biotechnol. 21:255-261). In plants, N-linked glycans may contain antigenic (Faye et al. (1993) Anal. Biochem. 109:104-108) and/or allergenic (van Ree et al. (2000) J. Biol. Chem. 275:11451-11458) β(1,2)-xylose (Xyl) residues attached to the N-linked Mannose of the glycan core and α(1,3)-fucose (Fuc) residues linked to the proximal GlcNAc that are not present on mammalian glycans. Plant glycans, however, do not contain sialic acid residues and plant antibodies do not require these residues for successful topical passive immunization (Ma et al., supra; Zeitlin et al., supra).

Glycosylation processing in the endoplasmic reticulum (ER) is conserved amongst almost all species and restricted to oligomannose (Man₅₋₉GlcNAc₂) type N-glycans, whereas the Golgi-generated processing to hybrid and complex type glycans is highly diverse (Helenius et al. (2001) Science 291:2364-2369). When attached to the C-terminus, the ER retrieval motif, KDEL, allows glycoproteins to be retained in, or returned to, the ER. Although there are exceptions (Navazio et al. (2002) Biochemistry 41:14141-14149), in general glycans attached to proteins containing a C-terminal KDEL sequence would be expected to be restricted mainly to the oligomannose type N-glycans (Helenius et al. (2001) Science 291:2364-2369; Henderson et al. (1997) Planta 202:313-323; Bauly et al. (2000) Plant Physiol. 124:1229-1238).

ER retention of expressed proteins in transgenic plants usually improves the production levels (Conrad et al. (1998) Plant Mol. Biol. 38: 101-109; Sharp et al. (2001) Biotechnol. Bioeng. 73:338-346). However, since glycan processing can affect the stability of antibodies (Rudd et al. (2001) Science 291:2370-2376), it is unclear whether a MAb^{P} with modified glycan structures would be active and able to confer effective systemic post-exposure prophylaxis.

It has been shown that the inclusion of KDEL or HDEL amino acid sequences at the carboxy terminus of at least one protein enhanced the recognition for that protein by the plant endoplasmic reticulum retention machinery. See, Munro and Pelham (1987) Cell 48:988-997; Denecke et al. (1991) EMBO-J. 11:2345; Herman et al. (1991) Planta 182:305; and Wandelt et al. (1992) The Plant Journal 2:181, each of which is incorporated herein by reference in its entirety.

Chimeric antibody technology, such as that used for the antibodies described in this invention, bridges both hybridoma and genetic engineering technologies to provide reagents, as well as products derived therefrom, for the treatment and diagnosis of human cancer.

The chimeric antibodies of the present invention embody a combination of the advantageous characteristics of MAbs. Like mouse MAbs, they can recognize and bind to a tumor antigen present in cancer tissue; however, unlike mouse MAbs, the “human-specific” properties of the chimeric antibodies lower the likelihood of an immune response to the antibodies, and result in prolonged survival in the circulation through reduced clearance. Moreover, using the methods disclosed herein, any desired antibody isotype can be combined with any particular antigen combining site.

The invention, as disclosed and described herein, overcomes the prior art problems with plant-derived antibodies by optimizing factors related to gene regulatory elements in plants and stable expression of antibodies in transgenic plants. The invention provides methods and compositions for the production of anti-tumor plant derived antibodies for use as therapeutics against cancer.

### III. SUMMARY OF THE INVENTION

The invention, as disclosed and described herein, provides methods and compositions for treating, ameliorating, or detecting human cancers.

In one aspect, the invention provides a plant-derived human monoclonal antibody that binds a human carcinoma antigen. The antigen may be presented in a cancer cell line or cell in vivo.

In one embodiment, the plant-derived monoclonal antibody comprises CO17-1A MAb^{p}, wherein CO17-1A MAb^{p} binds a human colorectal carcinoma antigen in a cancer cell line, or in vivo.

In another embodiment, the human colorectal carcinoma antigen comprises Ag GA733-2, and the cell line is SW948.

In another embodiment, CO17-1A MAb^{p} contains predominantly oligomannose type N-glycans and has substantially reduced and preferably no α(1,3)-linked fucose residues. Substantially reduced α(1,3)-linked fucose residues refers to a concentration range of about 10% to 0% of α(1,3)-linked fucose residues, for example, about 8%, 6%, 4%, 2% of α(1,3)-linked fucose residues.

In yet another embodiment, the plant-derived monoclonal antibody is encoded by a polynucleotide molecule comprising SEQ ID NO: 1, SEQ ID NO: 3, or a combination thereof, or a polynucleotide molecule having a sequence that is substantially homologous to SEQ ID NO: 1, SEQ ID NO: 3, or a combination thereof.

In another embodiment, the plant-derived monoclonal antibody comprises a polypeptide molecule comprising SEQ ID NO: 2, SEQ ID NO: 4, or a combination thereof, or a polypeptide molecule having a sequence that is substantially homologous to SEQ ID NO: 2, SEQ ID NO: 4, or a combination thereof.

In another aspect, the invention provides an expression vector that comprises one or more gene constructs comprising polynucleotides encoding one or more CO17-1A MAb^{p} subunits under the control of one or more promoters, operatively linked to regulatory control elements and Agrobacterium T-DNA terminal repeats.

In one embodiment, the regulatory control elements comprise an alfalfa mosaic virus untranslated leader sequence, an ER retention signal such as KDEL, or both.

In another embodiment, CO17-1A MAb^{p} subunits comprise an immunoglobulin heavy chain, light chain, or both.

In another embodiment, the expression of the heavy chain, the light chain or both are under the control of one or more promoters. Preferably, the promoters are constitutive promoters comprising cauliflower mosaic virus 35S promoter with duplicated upstream B domains, and a potato proteinase inhibitor II promoter.

In a preferred embodiment, the expression vector is pBICO17.

In yet another aspect, the invention provides a transgenic plant that expresses CO17-1A MAb^{p}, or a subunit thereof.

In a preferred embodiment the transgenic plant is a transgenic tobacco plant transformed with an expression vector comprising pBICO17.

In another aspect, the invention provides a pharmaceutical composition for treating, ameliorating, or detecting a human cancer comprising a pharmaceutically effective amount of a CO17-1A MAb^{p}, and an acceptable carrier or diluent.

In yet another aspect, the invention provides a diagnostic test kit for detection of human cancer comprising CO17-1A MAb^{p}, or a polynucleotide molecule encoding one or more subunits of the CO17-1A MAb^{p}, and a detection agent comprising a detectable label.

In another aspect, the invention provides methods for treating or ameliorating the burden of cancer comprising administering to a mammal, inclusive of humans, in need thereof an effective amount of the pharmaceutical composition of the invention.

These and other aspects and embodiments of the invention are disclosed in detail herein.

### IV. BRIEF DESCRIPTION OF THE FIGURES

FIG. 1. Heavy chain (HC) and light chain (LC) genes in a plant binary vector for Agrobacterium-mediated transformation. The T-DNA region was transferred to tobacco using A. tumefaciens EHA105. Pin2p and Pin2t: promoter and terminator of potato proteinase inhibitor II (Pin2) gene from potato, respectively; LC: cDNA of LC of CO17-1A; 35Sp: cauliflower mosaic virus ³⁵S promoter with duplicated upstream B domain; AMV: untranslated leader sequence of alfalfa mosaic virus RNA4; HC: cDNA of HC of CO17-1A; NOSt: terminator of nopaline synthase (NOS) gene. This binary vector contains the nptII gene under the control of the NOS promoter for a selectable marker to confer resistance to antibiotic kanamycin. I. LC expression cassette from pGEMPinLC. II. HC expression cassette from pBI525HC. Arrow sets and bars indicate the PCR primer sets for HC and LC gene, and the sequenced region, respectively.

FIG. 2. A. Western blot of LC expression under the control of Pin2 promoter in transgenic tobacco. Western blot to compare LC expression between leaves before, and 1, 24 and 48 h after mechanical wounding. Lanes 1, 2, 3, and 4: 10 μl of leaf extract of transgenic line before, and 1, 24, and 48 h after wounding, respectively; Lane 5 and 6: 10 μl of leaf extract of non-transgenic leaf before, and 48 h after wounding, respectively; Lane 7:20 ng of purified CO17-1A antibody from hybridoma. For wounding, leaves of tissue cultured plants were crushed with tissue forceps (Aesculap #BD-591, Burlingame, Calif.). LC is the light chain (25 kDa) of MAb CO17-1A. B. Western blots of LC and HC of MAb CO17-1A in transgenic tobacco. I. HC western blot. II. LC western blot. Lanes 1, 2, and 3: transgenic tobacco lines T1, T2, and T3, respectively; Lane 4: non-transgenic tobacco; Lane 5: 2 BenchMark Prestained Protein Ladder (Invitrogen, San Diego, Calif.); Lane 6: Blank, Lane 7: 10 ng of MAb CO17-1A from hybridoma, respectively. Upper and lower arrows indicate HC (50 kDa) and LC (25 kDa) proteins, respectively. 10 μl of leaf extracts (0.2 mg of leaf fresh weight/μl) were loaded.

FIG. 3. Binding activity of plant expressed MAbCO17-1A for Ag GA733-2E by ELISA. The ELISA assay was conducted using the 96 well plates coated with 2 μg/ml of the Ag GA733-2E. CO17-1A: 50 μl of 2.0 μg/ml of 10 the purified MAb CO17-1A from the hybridoma supernatant; T1, T2, and T3:50 μl of leaf extracts of tobacco transgenic lines T1, T2, and T3 producing the HC and LC bands; NT: 50 μl of leaf extracts of non-transgenic tobacco line. Statistical significance of immunological data was calculated with Student's t test using MINITAB™ statistical software (Minitab Inc., State College, Pa.) indicates significantly more value of transgenic lines or purified MAb CO17-1A compared to non-transgenic line (p=0.05).

### V. DETAILED DESCRIPTION OF THE INVENTION

The invention, as disclosed and described herein, provides plant-derived monoclonal antibodies that have applicability in the treatment and diagnosis of human cancer.

The invention provides anti-tumor monoclonal antibodies in plants through the use of plant expression vectors containing one or more T-DNA constructs harboring polynucleotide molecules encoding antibody genes placed under the control of one or more promoters.

In particular, the invention provides polynucleotide molecules encoding antibodies, including antibody subunits and fragments thereof. Antibodies of the invention comprises immunoglobulin chains including, for example, a human C region and a non-human, V region. The immunoglobulin chains include, H chain (HC), L chain (LC), or both.

The invention also provides individual H and L immunoglobulin chains, as well as complete assembled molecules having human L and H chains with specificity for human tumor cell antigens, wherein HC and LC have the same or different binding specificity with the antigen.

Among other immunoglobulin chains and/or molecules provided by the invention are antibodies with monovalent, bivalent or multivalent specificity for a tumor cell antigen, i.e., a complete, functional immunoglobulin molecule comprising: H chain and L chain, one or both chains comprising a V region with anti-tumor cell specificity, and antibody subunits such as Fab, Fab′, and F(ab′)₂.

The polynucleotides of the invention encoding LC, HC or both are placed under the control of one or more different or same promoters comprising inducible promoters, constitutive promoters, or both. In a preferred embodiment, polynucleotides encoding LC and HC are placed under constitutive promoters comprising potato proteinase inhibitor II (pin 2p) and constitutive duplicated CaMV 35S promoter (Ca2p), respectively.

The plant-derived antibody according to the invention includes truncated and/or N-terminally or C-terminally extended forms of the antibody, analogs having amino acid substitutions, additions and/or deletions, allelic variants and derivatives of the antibody, so long as their sequences are substantially homologous to the native human or mammalian-derived antibody and have specificity to an antigen bound by a human or mammalian monoclonal antibody. In particular, the plant derived antibody according to the invention comprises modifications to the N- or C-terminal ends of one or all immunoglobulin chains, which modifications can comprise one or more regulatory control elements or the addition of an ER retention signal. Suitable ER retention signals include the Lys-Asp-Glu-Leu (KDEL; SEQ ID NO: 9)) and His-Asp-Glu-Leu (HDEL; SEQ ID NO: 10) tetrapeptides.

The C-terminal ends of immunoglobulin chains comprising the plant-derived antibodies of the invention can, for example, be modified with ER retention signals by including the nucleic acid coding sequences for such signals in one of the PCR primers used to produce the coding sequences for the immunoglobulin chains. In one embodiment, the nucleic acid sequence 5′-GAGCTCATCTTT-3′ (SEQ ID NO: 11) can be included in the reverse PCR primer used to amplify an immunoglobulin chain nucleic acid sequence. The inclusion of SEQ ID NO: 11 in a reverse PCR primer will place codons encoding the KDEL sequence on the 3′-end of the amplified immunoglobulin nucleic acid sequence. See, e.g., Ko et al., Proc. Nat. Acad. Sci. USA 100: 8013-8018, the entire disclosure of which is herein incorporated by reference.

In a preferred embodiment, a plant-derived antibody of the invention of the invention comprises an alfalfa mosaic virus untranslated leader sequence and a Lys-Asp-Glu-Leu (KDEL) endoplasmic reticulum retention signal operably attached to the N- and C-termini of the immunoglobulin heavy chain, respectively.

Definitions

The definitions used in this application are for illustrative purposes and do not limit the scope of the invention.

As used herein, the term “plant” refers to whole plants, plant organs (i.e., leaves, stems, flowers, roots, etc.), seeds and plant cells (including tissue culture cells), and progeny of same. The class of plants that can be used in the method of the invention is generally as broad as the class of higher plants amenable to transformation techniques, including both monocotyledonous and dicotyledonous plants, as well as certain lower plants such as algae. Suitable plants include plants of a variety of ploidy levels, including polyploid, diploid and haploid. The term “transgenic plant” refers to a plant modified to express one or more antibody genes.

As used herein, the term “gene” refers to an element or combination of elements that are capable of being expressed in a cell, either alone or in combination with other elements. In general, a gene comprises (from the 5′ to the 3′ end): (1) a promoter region, which includes a 5′ nontranslated leader sequence capable of functioning in plant cells; (2) a structural gene or polynucleotide sequence, which codes for the desired protein; and (3) a 3′ nontranslated region, which typically causes the termination of transcription and the polyadenylation of the 3′ region of the RNA sequence. Each of these elements is operably linked by sequential attachment to the adjacent element. A gene comprising the above elements is inserted by standard recombinant DNA methods into a plant expression vector.

As used herein, “promoter” refers to a region of a DNA sequence active in the initiation and regulation of the expression of a structural gene. This sequence of DNA, usually upstream to the coding sequence of a structural gene, controls the expression of the coding region by providing the recognition for RNA polymerase and/or other elements required for transcription to start at the correct site.

As used herein, “protein” is used interchangeably with polypeptide, peptide and peptide fragments.

As used herein, “polynucleotide” includes cDNA, RNA, DNA/RNA hybrid, anti-sense RNA, ribozyme, genomic DNA, synthetic forms, and mixed polymers, both sense and antisense strands, and may be chemically or biochemically modified to contain non-natural or derivatized, synthetic, or semi-synthetic nucleotide bases. Also, included within the scope of the invention are alterations of a wild type or synthetic gene, including but not limited to deletion, insertion, substitution of one or more nucleotides, or fusion to other polynucleotide sequences, provided that such changes in the primary sequence of the gene do not alter the expressed peptide ability to elicit passive immunity.

As used herein, “gene products” include any product that is produced in the course of the transcription, reverse-transcription, polymerization, translation, post-translation and/or expression of a gene. Gene products include, but are not limited to, proteins, polypeptides, peptides, peptide fragments, or polynucleotide molecules.

As disclosed herein, “substantially homologous sequences” include those sequences which have at least about 50% homology, preferably at least about 60%, more preferably at least about 70% homology, even more preferably at least about 80% homology, and most preferably at least about 95% or more homology to the polynucleotides of the invention.

As used herein, “polypeptides” include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptide, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, “antibody” refers to intact molecules as well as to fragments thereof, such as Fab, F(ab′)₂, and Fv fragments, which are capable of binding an epitopic determinant. Antibody fragments refer to antigen-binding immunoglobulin peptides which are at least about 5 to about 15 amino acids or more in length, and which retain some biological activity or immunological activity of an immunoglobulin.

As used herein, the term “monoclonal antibody” includes antibodies which display a single binding specificity and affinity for a particular epitope. These antibodies are plant-derived or mammalian-derived antibodies, including murine, human and humanized antibodies. The term “human monoclonal antibody” as used herein, refers to antibodies displaying a single binding specificity which have variable and constant regions derived from human germ-line immunoglobulin sequences.

I. CO17-1A MAb^{p}

In one aspect, the invention provides an anti-tumor monoclonal antibody, CO17-1A MAb^{p}, that binds a human colorectal carcinoma antigen. CO17-1A MAb has been studied for colorectal cancer therapeutic research and has been reported to be relatively efficacious in the treatment of micrometastases and minimal residual disease (Riethmuller et al. (1994) Lancet 343:1177-1183; and Stoger et al. (2002) Curr. Opin. Biotechnol. 13:161-166), each of which is incorporated herein by reference in its entirety.

CO17-1A MAb^{p} is expressed and fully assembled in plants without any gene silencing. For the example, the concentration of CO17-1A MAb^{p} in the plant is in the range of about 0.01% to 5%, for example, 0.07%, 0.1%, 1%, 2.5%, or 5% of the total soluble protein in plants. It is intended herein that by recitation of such specified ranges, the ranges recited also include all those specific integer amounts between the recited ranges. For example, in the range about 0.1 to 1%, it is intended to also encompass 0.2, 0.3, 0.4, 0.5, 0.6, etc.

In one embodiment, CO17-1A MAb^{p} of the invention exhibited structural differences as compared to their mammalian or plant-derived counterparts. Structural differences in proteins expressed in heterologous systems are known to arise from posttranslational modifications, mostly from glycosylation. For example, CO17-1A MAb^{p} contained predominantly oligomannose type N-glycans and had substantially reduced or no potentially antigenic α(1,3)-linked fucose residues. Differences in N-glycosylation do not affect the efficacy of the antibody.

In another embodiment CO-17-1A MAb^{p} was modified to contain a KDEL sequence. This MAb^{p}, displays predominantly oligomannose type N-glycans, for example, about 70%, 80%, 90%, 95% or more oligomannose type N-glycans can be identified.

The presence of Man₆₋₉GlcNAc₂ (about 70-95%, preferably 90%), GlcNAc₂Man₃GlcNAc₂ (about 3-6% preferably about 4.3%) and GlcNAc₂(Xyl)Man₃GlcNAc₂ (about 3-7%, preferably about 5.7%) glycans in MAb^{P} indicates that most of MAb^{P}/KDEL did not pass further along the secretory pathway than the cis-Golgi stack, from which it was probably retrieved and returned to the ER (Henderson et al. (1997) Planta 202:313-23; and Bauly et al.(2000) Plant Physiol. 124:1229-1238, each of which is incorporated herein by reference in its entirety). As a result, the modified MAb^{P} did not contain glycans with the plant specific α(1,3)-linked Fuc residues. This in turn minimized the risk of immunogenic and allergenic reactions to this epitope in humans.

The α(1,3)-linked Fuc residue is recognized by both IgG and IgE (Wilson et al. (1998) Glycobiology 8:651-661, incorporated herein by reference in its entirety). If present, the xylose residue that is α(1,2)-linked to the β-linked core mannose of the sugars attached to MAb^{P} forms part of the anti-α(1,3)-linked Fuc antibody epitope, but does not on its own constitute a potent epitope. Moreover, the xylose-containing glycans in MAb^{P} are also known to contain an α1,3-antenna and, on these grounds too, the xylose is unlikely to bind IgE. In contrast, α-Gal residues are known to be potent antigens.

2. Plant-Derived Antibodies, and Antibodies Subunits and Fragments Thereof

The invention provides plant-derived human, humanized or chimeric antibodies, including antibody subunits and fragments thereof, with specificity to human tumor antigens. The antibodies of the invention include antibodies that are expressed and isolated by recombinant means from a transgenic plant.

In one embodiment, the antibodies include immunoglobulin molecules having H and L chains associated so that the overall molecule exhibits the desired antigen binding and recognition properties. Various types of immunoglobulin molecules are provided: monovalent, divalent, multivalent, or molecules with the specificity-determining V binding domains attached to moieties carrying desired functions, among others.

In another embodiment, the invention provides for fragments of chimeric immunoglobulin molecules such as Fab, Fab′, or F(ab′)₂ molecules or those proteins encoded by truncated genes to yield molecular species functionally resembling these fragments. A chimeric immunoglobulin molecule comprises a chimeric chain containing a constant (C) region substantially similar to that present in a natural human immunoglobulin, and a variable (V) region having the desired anti-tumor specificity of the invention. Antibodies having chimeric H chains and L chains of the same or different V region binding specificity are prepared by appropriate association of the desired polypeptide chains.

The immunoglobulin molecules are encoded by genes which include the kappa, lambda, alpha, gamma, delta, epsilon or mu constant regions, as well as any number of immunoglobulin variable regions. Light chains are classified as either kappa or lambda. Light chains comprise a variable light (V_{L}) and a constant light (C_{L}) domain. Heavy chains are classified as gamma, mu, alpha, delta, or epsilon, which in turn define the immunoglobulin classes IgG, IgM, IgA, IgD and IgE, respectively. Heavy chains comprise variable heavy (V_{H}), constant heavy 1 (CH1), hinge, constant heavy 2 (CH2), and constant heavy 3 (CH3) domains. The human IgG heavy chains are further sub-classified based on their sequence variation, and the subclasses are designated IgG1, IgG2, IgG3 and IgG4.

Antibodies comprise two pairs of a light and heavy domain. The paired V_{L} and V_{H} domains each comprise a series of seven subdomains: framework region 1 (FR1), complementarity determining region 1 (CDR1), framework region 2 (FR2), complementarity determining region 2 (CDR2), framework region 3 (FR3), complementarity determining region 3 (CDR3), and framework region 4 (FR4) which constitute the antibody-antigen recognition domain.

In general, as used herein, the term plant-derived antibody or plant-derived monoclonal antibody (MAb^{p}) encompasses a variety of modifications, particularly those that are present in polypeptides expressed by polynucleotides in a host cell. It will be appreciated that polypeptides often contain amino acids other than the 20 amino acids commonly referred to as the naturally occurring amino acids, and that many amino acids, including the terminal amino acids, may be modified in a given polypeptide, either by natural processes, such as processing and other post-translational modifications, or by chemical modification techniques.

Modifications occur anywhere in a polypeptide, including the peptide backbone, the amino acid side chains and the amino or carboxyl termini. Blockage of the amino or carboxyl group in a polypeptide, or both, by a covalent modification, occur in natural or synthetic polypeptides. Such modifications may be present in the antibody polypeptides of the present invention, as well. In general, the nature and extent of the modifications are determined by the host cell's post-translational modification capacity and the modification signals present in the polypeptide amino acid sequence. It will be appreciated that the same type of modification may be present in the same or varying degrees at several sites in a polypeptide.

The plant-derived antibody according to the invention includes truncated and/or N-terminally or C-terminally extended forms of the antibody, analogs having amino acid substitutions, additions and/or deletions, allelic variants and derivatives of the antibody, so long as their sequences are substantially homologous to the native human or mammalian-derived antibody and have specificity to an antigen bound by a human or mammalian monoclonal antibody.

Variations in the structure of plant-derived antibodies may arise naturally as allelic variations, as disclosed above, due to genetic polymorphism, for example, or may be produced by human intervention (i.e., by mutagenesis of cloned DNA sequences), such as induced point, deletion, insertion and substitution mutants. Minor changes in amino acid sequence are generally preferred, such as conservative amino acid replacements, small internal deletions or insertions, and additions or deletions at the ends of the molecules.

Substitutions may be designed based on, for example, the model of Dayhoff et al. (1978) Atlas of Protein Sequence and Structure, Natl. Biomed. Res. Found. Washington, D.C. These modifications can result in changes in the amino acid sequence, provide silent mutations, modify a restriction site, or provide other specific mutations.

The conserved and variable sequence regions of a plant-derived antibody and the homology thereof can be determined by techniques known to the skilled artisan, such as sequence alignment techniques. For example, the determination of percent identity between two sequences can also be accomplished using a mathematical algorithm, as described below.

3. Plant Expression Vectors

Also encompassed within the scope of the invention are plant expression vectors containing the gene constructs of the invention. Expression vectors are DNA sequences that are required for the transcription of cloned copies of genes and the translation of their mRNAs in an appropriate host. Such expression vectors are used to express eukaryotic and prokaryotic genes in plants. Expression vectors include, but are not limited to, cloning vectors, modified cloning vectors, specifically, designed plasmids or viruses.

According to one embodiment of the invention, there are provided plant expression vectors containing one or more gene constructs of the invention carrying the antibody genes, including antibody subunit genes or fragments thereof. The plant expression vectors of the invention contain the necessary elements to accomplish genetic transformation of plants so that the gene constructs are introduced into the plant's genetic material in a stable manner, i.e., a manner that will allow the antibody genes to be passed onto the plant's progeny. The design and construction of the expression vectors influence the integration of the gene constructs into the plant genome and the ability of the antibody genes to be expressed by plant cells.

Preferred among expression vectors are vectors carrying a functionally complete human or mammalian heavy or light chain sequence having appropriate restriction sites engineered so that any variable V_{H} or variable V_{L} chain sequence with appropriate cohesive ends can be easily inserted therein. Human C_{H} or C_{L} chain sequence-containing vectors are thus an embodiment of the invention and can be used as intermediates for the expression of any desired complete H or L chain in any appropriate host.

Many vector systems are available for the expression of cloned HC and LC genes in host cells. Different approaches can be followed to obtain complete HC and LC subunit antibodies. In one embodiment, HC and LC were co-expressed in the same cells to achieve intracellular association and linkage of HC and LC into complete tetrameric HC and LC antibodies. The co-expression can occur by using either the same or different plasmids in the same host.

Polynucleotides encoding both HC and LC are placed under the control of one or more different or the same promoters, for example in the form of a dicistronic operon, into the same or different expression vectors. The expression vectors are then transformed into cells, thereby selecting directly for cells that express both chains.

In one embodiment, the polynucleotide encoding LC and polynucleotides encoding HC are present on two mutually compatible expression vectors which are each under the control of different or the same promoter(s). In this embodiment, the expression vectors are co-transformed or transformed individually. For example, cells are transformed first with an expression vector encoding one chain, for example LC, followed by transformation of the resulting cell with an expression vector encoding a HC.

In a preferred embodiment, a single expression vector carrying polynucleotides encoding both the HC and LC is used. Cell lines expressing HC and LC molecules could be transformed with expression vectors encoding additional copies of LC, HC, or LC plus HC in conjunction with additional selectable markers to generate cell lines with enhanced properties, such as higher production of assembled HC and LC antibody molecules or enhanced stability of the transformed cell lines.

Specifically designed expression vectors allow the shuttling of DNA between hosts, such as between bacteria and plant cells. According to a preferred embodiment of the invention, the expression vector contains an origin of replication for autonomous replication in host cells, selectable markers, a limited number of useful restriction enzyme sites, active promoter(s), and additional regulatory control sequences.

Preferred among expression vectors, in certain embodiments, are those expression vectors that contain cis-acting control regions effective for expression in a host operatively linked to the polynucleotide of the invention to be expressed. Appropriate trans-acting factors are supplied by the host, supplied by a complementing vector or supplied by the vector itself upon introduction into the host.

In certain preferred embodiments in this regard, the expression vectors provide for specific expression. Such specific expression is an inducible expression, cell or organ specific expression, host-specific expression, or a combination thereof.

In a preferred embodiment of the invention, the plant expression vector is an Agrobacterium-based expression vector. Various methods are known in the art to accomplish the genetic transformation of plants and plant tissues by the use of Agrobacterium-mediated transformation systems, i.e., A. tumefaciens and A. rhizogenesis. Agrobacterium is the etiologic agent of crown gall, a disease of a wide range of dicotyledons and gymnosperms that results in the formation of tumors or galls in plant tissue at the site of infection. Agrobacterium, which normally infects the plant at wound sites, carries a large extrachromosomal element called Ti (tumor-inducing) plasmid.

Ti plasmids contain two regions required for tumor induction. One region is the T-DNA (transferred-DNA) which is the DNA sequence that is ultimately found stably transferred to plant genomic DNA. The other region is the vir (virulence) region which has been implicated in the transfer mechanism. Although the vir region is absolutely required for stable transformation, the vir DNA is not actually transferred to the infected plant. Transformation of plant cells mediated by infection with A. tumefaciens and subsequent transfer of the T-DNA alone have been well documented. See, i.e., Bevan et al. (1982) Int. Rev. Genet. 16:357 incorporated herein by reference in its entirety.

A. rhizogenes has also been used as a vector for plant transformation. This bacterium, which incites root hair formation in many dicotyledonous plant species, carries a large extrachromosomal element called a Ri (root-inducing) plasmid which functions in a manner analogous to the Ti plasmid of A. tumefaciens. Transformation using A. rhizogenes has developed analogously to that of A. tumefaciens and has been successfully utilized to transform the plant of this invention.

Agrobacterium system has been developed to permit routine transformation of a variety of plant tissues. Representative tissues transformed by this technique include, but are not limited to, tobacco, tomato, sunflower, cotton, rapeseed, potato, poplar, and soybean, among others.

3.1. Promoters

Promoters are responsible for the regulation of the transcription of DNA into mRNA. A number of promoters which function in plant cells are known in the art, and may be employed in the practice of the present invention. These promoters are obtained from a variety of sources such as, for example, plants or plant viruses, bacteria, among others.

The invention, as described and disclosed herein, encompasses the use of constitutive promoters, inducible promoters, or both.

In general, an “inducible promoter” is a promoter that is capable of directly or indirectly activating transcription of one or more DNA sequences or genes in response to an inducer. In the absence of an inducer the DNA sequences or genes will not be transcribed. Typically the protein factor, that binds specifically to an inducible promoter to activate transcription, is present in an inactive form which is then directly or indirectly converted to the active form by the inducer. The inducer can be a chemical agent such as a protein, metabolite, growth regulator, herbicide or phenolic compound or a physiological stress imposed directly by heat, cold, wound, salt, or toxic elements, light, desiccation, pathogen infection, or pest-infestation.

Inducible promoters are determined using any methods known in the art. For example, the promoter may be operably associated with an assayable marker gene such as GUS (glucouronidase), the host plant can be engineered with the construct; and the ability and activity of the promoter to drive the expression of the marker gene in the harvested tissue under various conditions assayed.

A plant cell containing an inducible promoter is exposed to an inducer by externally applying the inducer to the cell or plant such as by spraying, harvesting, watering, heating or similar methods. In addition, inducible promoters include tissue specific promoters that function in a tissue specific manner to regulate the gene of interest within selected tissues of the plant. Examples of such tissue specific promoters include seed, flower or root specific promoters as are well known in the field.

A “constitutive promoter” is a promoter that directs the expression of a gene throughout the various parts of a plant and continuously throughout plant development.

In one embodiment of the invention, promoters are tissue-specific. Non-tissue-specific promoters (i.e., those that express in all tissues after induction), however, are preferred. More preferred are promoters that additionally have no or very low activity in the uninduced state. Most preferred are promoters that additionally have very high activity after induction. Particularly preferred among inducible promoters are those that can be induced to express a protein by environmental factors that are easy to manipulate.

In a preferred embodiment of the invention, one or more constitutive promoters are used to regulate expression of antibody genes or antibody subunit genes in a plant.

Examples of an inducible and/or constitutive promoters include, but are not limited to, promoters isolated from the caulimovirus group such as the cauliflower mosaic virus 35S promoter (CaMV35S), the enhanced cauliflower mosaic virus 35S promoter (enh CaMV35S), the figwort mosaic virus full-length transcript promoter (FMV35S), the promoter isolated from the chlorophyll a/b binding protein, proteinase inhibitors (PI-I, PI-II), defense response genes, phytoalexin biosynthesis, phenylpropanoid phytoalexin, phenylalanine ammonia lyase (PAL), 4-coumarate CoA ligase (4CL), chalcone synthase (CHS), chalcone isomerase (CHI), resveratrol (stilbene) synthase, isoflavone reductase (IFR), terpenoid phytoalexins, HMG-CoA reductase (HMG), casbene synthetase, cell wall components, lignin, phenylalanine ammonia lyase, cinnamyl alcohol dehydrogenase (CAD), caffeic acid o-methyltransferase, lignin-forming peroxidase, hydroxyproline-rich glycoproteins (HRGP), glycine-rich proteins (GRP), thionins, hydrolases, lytic enzymes, chitinases (PR-P, PR-Q), class I chitinase, basic, Class I and II chitinase, acidic, class II chitinase, bifunctional lysozyme, β-1,3-Glucanase, arabidopsis, β-fructosidase, superoxide dismutase (SOD), lipoxygenase, prot., PR1 family, PR2, PR3, osmotin, PR5, ubiquitin, wound-inducible genes, win1, win2 (hevein-like), wun1, wun2, nos, nopaline synthase, ACC synthase, HMG-CoA reductase hmgl, 3-deoxy-D-arabino-heptulosonate-7-phosphate synthase, HSP7033, Salicylic acid inducible, acid peroxidase, PR-proteins, glycine-rich protein, methyl jasmonate inducible, vspB⁴², heat-shock genes, HSP70, cold-stress inducible, drought, salt stress, hormone inducible, gibberellin, α-amylase, abscisic acid, EM-1, RAB, LEA genes, ethylene, phytoalexin biosyn genes, or a combination thereof.

The above-noted promoters are listed solely by way of illustration of the many commercially available and well known plant promoters that are available to those of skill in the art for use in accordance with this aspect of the present invention. It will be appreciated that any other plant promoter suitable for, for example, introduction, maintenance, propagation or expression of a polynucleotide or polypeptide of the invention in plants may be used in this aspect of the invention.

3.3. Regulatory Control Elements

Gene constructs of the present invention can also include other optional regulatory elements that regulate, as well as engender, expression. Generally such regulatory control elements operate by controlling transcription. Examples of such regulatory control elements include, for example, enhancers (either translational or transcriptional enhancers as may be required), repressor binding sites, terminators, leader sequences, and the like.

Specific examples of these elements include, but are not limited to, the enhancer region of the 35S regulatory region, as well as other enhancers obtained from other regulatory regions, and/or the ATG initiation codon and adjacent sequences. The initiation codon must be in phase with the reading frame of the coding sequence to ensure translation of the entire sequence. The translation control signals and initiation codons are from a variety of origins, both natural and synthetic. Translational initiation regions are provided from the source of the transcriptional initiation region, or from the structural gene. The sequence is also derived from the promoter selected to express the gene, and can be specifically modified to increase translation of the mRNA.

The nontranslated leader sequence is derived from any suitable source and is specifically modified to increase the translation of the mRNA. In one embodiment, the 5′ nontranslated region is obtained from the promoter selected to express the gene, the native leader sequence of the gene, coding region to be expressed, viral RNAs, suitable eucaryotic genes, or a synthetic gene sequence, among others.

In another embodiment, gene constructs of the present invention comprise a 3U untranslated region. A 3U untranslated region refers to that portion of a gene comprising a DNA segment that contains a polyadenylation signal and any other regulatory signals capable of effecting mRNA processing or gene expression. The polyadenylation signal is usually characterized by effecting the addition of polyadenylic acid tracks to the 3U end of the mRNA precursor.

The termination region or 3′ nontranslated region is employed to cause the termination of transcription and the addition of polyadenylated ribonucleotides to the 3′ end of the transcribed mRNA sequence. The termination region may be native with the promoter region, native with the structural gene, or may be derived from the expression vector or another source, and would preferably include a terminator and a sequence coding for polyadenylation. Suitable 3′ nontranslated regions of the chimeric plant gene include, but are not limited to: (1) the 3′ transcribed, nontranslated regions containing the polyadenylation signal of Agrobacterium tumor-inducing (Ti) plasmid genes, such as the nopaline synthase (NOS) gene, and (2) plant genes like the soybean 7S storage protein genes and the pea small subunit of the ribulose 1,5-bisphosphate carboxylase-oxygenase, among others.

The addition of appropriate introns and/or modifications of coding sequences for increased translation can also substantially improve foreign gene expression. Appropriate introns include, but are not limited to, the maize hsp70 intron, maize adh 1 intron, and rice actin intron.

In one embodiment, the regulatory control elements of the invention include an alfalfa mosaic virus untranslated leader sequence, an ER retention signal KDEL, or both.

3.4. Selectable Markers

To aid in identification of transformed plant cells, the gene constructs of this invention may be further manipulated to include selectable marker genes that are functional in bacteria, plants or both. Useful selectable markers include, but are not limited to, enzymes which provide for resistance to an antibiotic such as ampicillin resistance gene (Amp^{r}), tetracycline resistance gene (Tc^{r}), cycloheximide-resistance L41 gene, the gene conferring resistance to antibiotic G418 such as the APT gene derived from a bacterial transposon Tn903, the antibiotic hygromycin B-resistance gene, gentamycin resistance gene, and/or kanamycine resistance gene, among others. Similarly, enzymes providing for production of a compound identifiable by color change such as GUS, or luminescence, such as luciferase are included herein.

A selectable marker gene is used to select transgenic plant cells of the invention, which transgenic cells have integrated therein one or more copies of the gene construct of the invention. The selectable or screenable genes provides another control for the successful culturing of cells carrying the genes of interest. Transformed plant calli may be selected by growing the cells on a medium containing, for example, kanamycin.

4. Transformation Strategies

Host plants are genetically transformed to incorporate one or more gene constructs of the invention. There are numerous factors which influence the success of plant transformation. The design and construction of the expression vector influence the integration of the foreign genes into the genome of the host plant and the ability of the foreign genes to be expressed by plant cells. The type of cell into which the gene construct is introduced must, if whole plants are to be recovered, be of a type which is amenable to regeneration, given an appropriate regeneration protocol.

The integration of the polynucleotides encoding the desired gene into the plant host is achieved through strategies that involve, for example, insertion or replacement methods. These methods involve strategies utilizing, for example, direct terminal repeats, inverted terminal repeats, double expression cassette knock-in, specific gene knock-in, specific gene knock-out, random chemical mutagenesis, random mutagenesis via transposon, and the like. The expression vector is, for example, flanked with homologous sequences of any non-essential plant genes, bacteria genes, transposon sequence, or ribosomal genes. Preferably the flanking sequences are T-DNA terminal repeat sequences. The DNA is then integrated in host by homologous recombination occurred in the flanking sequences using standard techniques.

In a preferred embodiment of the invention, Agrobacterium-based transformation strategy is employed to introduce the gene constructs into plants. Such transformations preferably use binary Agrobacterium T-DNA vectors (Bevan (1984) supra) and the co-cultivation procedure (Horsch et al. (1985) Science 227:1229-1231, incorporated herein by reference in its entirety). Generally, the Agrobacterium transformation system is used to engineer dicotyledonous plants. The Agrobacterium transformation system may also be used to transform as well as transfer DNA to monocotyledonous plants and plant cells. See, for example, Hemalsteen et al. (1984) EMBO J. 3:3039-3041; Hooykass-Van Slogteren et al. (1984) Nature 311:763-764; Grimsley et al. (1987) Nature 325:1677-179; Boulton et al. (1989) Plant Mol. Biol. 12:31-40; Gould et al. (1991) Plant Physiol. 95:426-434, each of which is incorporated herein by reference in its entirety.

In other embodiments, various alternative methods for introducing recombinant nucleic acid constructs into plants and plant cells are utilized. These other methods are particularly useful where the target is a monocotyledonous plant or plant cell. Alternative gene transfer and transformation methods include, but are not limited to, protoplast transformation through calcium-polyethylene glycol (PEG)- or electroporation-mediated uptake of naked DNA. See, for example, Paszkowski et al. (1984) EMBO J. 3:2717-2722, Potrykus et al. (1985) Molec. Gen. Genet. 199:169-177; Fromm et al. (1985) Proc. Nat. Acad. Sci. USA 82:5824-5828; and Shimamoto (1989) Nature 338:274-276, each of which is incorporated herein by reference in its entirety. Electroporation of plant tissues are also disclosed in D'Halluin et al. (1992) Plant Cell 4:1495-1505, incorporated herein by reference in its entirety. Additional methods for plant cell transformation include microinjection, silicon carbide mediated DNA uptake (see, for example, Kaeppler et al. (1990) Plant Cell Reporter 9:415-418), and microprojectile bombardment (see, for example, Klein et al. (1988) Proc. Nat. Acad. Sci. USA 85:4305-4309; and Gordon-Kamm et al. (1990) Plant Cell 2:603-618, each of which is incorporated herein by reference in its entirety.

In the case of direct gene transfer, the gene construct is transformed into plant tissue without the use of the Agrobacterium plasmids. Direct transformation involves the uptake of exogenous genetic material into plant cells or protoplasts. Such uptake may be enhanced by use of chemical agents or electric fields. The exogenous material may then be integrated into the nuclear genome. The early work with direct transfer was conducted in the Nicotiana tobacum (tobacco) where it was shown that the foreign DNA was incorporated and transmitted to progeny plants. Several monocot protoplasts have also been transformed by this procedure including maize and rice.

Liposome fusion has also been shown to be a method for transforming plant cells. Protoplasts are brought together with liposomes carrying the desired gene. As membranes merge, the foreign gene is transferred to the protoplasts.

Alternatively, exogenous DNA can be introduced into cells or protoplasts by microinjection. In this technique, a solution of the plasmid DNA or DNA fragment is injected directly into the cell with a finely pulled glass needle.

A more recently developed procedure for direct gene transfer involves bombardment of cells by micro-projectiles carrying DNA. In this procedure, commonly called particle bombardment, tungsten or gold particles coated with the exogenous DNA are accelerated toward the target cells. The particles penetrate the cells carrying with them the coated DNA. Microparticle acceleration has been successfully demonstrated to lead to both transient expression and stable expression in cells suspended in cultures, protoplasts, immature embryos of plants including but not limited to onion, maize, soybean, and tobacco.

In addition to the methods described above, a large number of methods are known in the art for transferring cloned DNA into a wide variety of plant species, including gymnosperms, angiosperms, monocots and dicots. Minor variations make these technologies applicable to a broad range of plant species.

5. Transgenic Plants

The invention further relates to transgenic plants, including whole plants, plant organs (i.e., leaves, stems, flowers, roots, etc.), seeds and plant cells (including tissue culture cells), and progeny of same that are transformed with a gene construct according to the invention.

Once plant cells have been transformed, there are a variety of methods for regenerating plants. The particular method of regeneration will depend on the starting plant tissue and the particular plant species to be regenerated. In general, transformed plant cells are cultured in an appropriate medium, which contain selective agents such as, for example, antibiotics, where selectable markers are used to facilitate identification of transformed plant cells. Once callus forms, embryo or shoot formation are encouraged by employing the appropriate plant hormones in accordance with known methods and the shoots transferred to rooting medium for regeneration of plants. The plants are then used to establish repetitive generations, either from seeds or using vegetative propagation techniques. The presence of a desired gene, or gene product, in the transformed plant may be determined by any suitable method known to those skilled in the art. Included in these methods are southern, northern, and western blot techniques, ELISA, and bioassays.

In recent years, it has become possible to regenerate many species of plants from callus tissue derived from plant explants. The plants which can be regenerated from callus include monocots, such as, but not limited to, corn, rice, barley, wheat, and rye, and dicots, such as, but not limited to, sunflower, soybean, cotton, rapeseed and tobacco.

6. Polynucleotides Encoding Antibody Polypeptides

This invention also encompasses polynucleotides that correspond to and code for the antibody polypeptides. Nucleic acid sequences are either synthesized using automated systems well known in the art, or derived from a gene bank.

It will be appreciated that a great variety of modifications have been made to DNA and RNA that serve many useful purposes known to those of skill in the art. The polynucleotides of the invention embrace chemically, enzymatically or metabolically modified forms of polynucleotides.

The polynucleotides of the present invention encode, for example, the coding sequence for the structural gene (i.e., antibody gene), and additional coding or non-coding sequences. Examples of additional coding sequences include, but are not limited to, sequences encoding a secretory sequence, such as a pre-, pro-, or prepro-protein sequences. Examples of additional non-coding sequences include, but are not limited to, introns and non-coding 5′ and 3′ sequences, such as the transcribed, non-translated sequences that play a role in transcription and mRNA processing, including splicing and polyadenylation signals, for example, for ribosome binding and stability of mRNA.

The polynucleotides of the invention also encode a polypeptide which is the mature protein plus additional amino or carboxyl-terminal amino acids, or amino acids interior to the mature polypeptide (when the mature form has more than one polypeptide chain, for instance). Such sequences play a role in, for example, processing of a protein from precursor to a mature form, may facilitating protein trafficking, prolonging or shortening protein half-life or facilitating manipulation of a protein for assay or production, among others. The additional amino acids may be processed away from the mature protein by cellular enzymes.

In sum, the polynucleotides of the present invention encode, for example, a mature protein, a mature protein plus a leader sequence (which may be referred to as a preprotein), a precursor of a mature protein having one or more prosequences which are not the leader sequences of a preprotein, or a preproprotein, which is a precursor to a proprotein, having a leader sequence and one or more prosequences, which generally are removed during processing steps that produce active and mature forms of the polypeptide.

The polynucleotides of the invention include “variant(s)” of polynucleotides, or polypeptides as the term is used herein. Variants include polynucleotides that differ in nucleotide sequence from another reference polynucleotide. Generally, differences are limited so that the nucleotide sequences of the reference and the variant are closely similar overall and, in many regions, identical. As noted below, changes in the nucleotide sequence of the variant my be silent. That is, they may not alter the amino acids encoded by the polynucleotide. Where alterations are limited to silent changes of this type, a variant will encode a polypeptide with the same amino acid sequence as the reference.

Changes in the nucleotide sequence of the variant may alter the amino acid sequence of a polypeptide encoded by the reference polynucleotide. Such nucleotide changes may result in amino acid substitutions, additions, deletions, fusions and truncations in the polypeptide encoded by the reference sequence. According to a preferred embodiment of the invention, there are no alterations in the amino acid sequence of the polypeptide encoded by the polynucleotides of the invention, as compared with the amino acid sequence of the wild type or mammalian derived peptide.

The present invention further relates to polynucleotides that hybridize to the herein described sequences. The term “hybridization under stringent conditions” according to the present invention is used as described by Sambrook et al. (1989) Molecular Cloning, A Laboratory Manual, Cold Spring Harbor Laboratory Press 1.101-1.104. Preferably, a stringent hybridization according to the present invention is given when after washing for an hour with 1% SSC and 0.1% SDC at 50° C., preferably at 55° C., more preferably at 62° C., most preferably at 68° C., a positive hybridization signal is still observed. A polynucleotide sequence which hybridizes under such washing conditions with the nucleotide sequence shown in any sequence disclosed herein or with a nucleotide sequence corresponding thereto within the degeneration of the genetic code is a nucleotide sequence according to the invention.

The polynucleotides of the invention include polynucleotide sequences that have at least about 50%, 65%, 70%, 75%, 80%, 85%, 90%, 95%, 98%, 99% or more nucleotide sequence identity to the polynucleotides or a transcriptionally active fragment thereof. To determine the percent identity of two amino acid sequences or two nucleic acid sequences, the sequences are aligned for optimal comparison purposes (i.e., gaps can be introduced in the sequence of a first amino acid or nucleic acid sequence for optimal alignment with a second nucleic acid sequence). The amino acid residue or nucleotides at corresponding amino acid or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical overlapping positions/total # of positions×100). In one embodiment, the two sequences are the same length.

The determination of percent identity between two sequences also can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST program of Altschul et al. (1990), J. Mol. Biol. 215:403-410. BLAST nucleotide searches can be performed with the NBLAST program, score=100, wordlength=12 to obtain nucleotide sequences homologous to a nucleic acid molecules of the invention. The BLAST protein searches can be performed with the XBLAST program, score=50, wordlength=3 to obtain amino acid sequences homologous to a protein molecule of the invention. To obtain gapped alignments for comparison purposes, Gapped BLAST can be utilized as described in Altschul et al. (1997) Nucleic Acids Res. 25:3389-3402.

Alternatively, PSI-Blast can be used to perform an iterated search which detects distant relationships between molecules (Id.). When utilizing BLAST, Gapped BLAST and PSI-Blast programs, the default parameters of the respective programs (i.e., XBLAST and NBLAST program can be used. Another preferred, non-limiting example of a mathematical algorithm utilized for the comparison of sequences is the algorithm of Myers and Miller (1988) CABIOS 4:11-17. Such an algorithm is incorporated into the ALIGN program (version 2.0) which is part of the GCG sequence alignment software package. When utilizing the ALIGN program for comparing amino acid sequences of a PAM 120 weight residue table, a gap length penalty of 12 and a gap penalty of 4 can be used. In an alternate embodiment, alignments can be obtained using the NA-MULTIPLE-ALIGNMENT 1.0 program, using a GapWeight of 5 and a GapLengthWeight of 1.

7. Methods of Using Plant-Derived Antibodies

In one aspect the invention as described herein provide methods for using the plant-derived antibodies. The plant-derived antibodies of the invention are used for therapeutic and/or diagnostic purposes by themselves, for example, acting via complement-mediated lysis and antibody-dependent cellular cytotoxicity, or coupled to other therapeutic moieties, such as ricin, radionuclides, drugs, etc. The antibodies may be advantageously utilized in combination with factors, such as lymphokines, colony-stimulating factors, and the like, which increase the number or activity of antibody-dependent effector cells.

In one embodiment, the plant-derived antibody of the invention, preferably having a human C region, is utilized for passive immunization, especially in humans, with reduced negative immune reactions such as serum sickness or anaphylactic shock, as compared to the mammalian-derived counterpart antibodies.

In yet another embodiment, the plant-derived antibody of the invention is used in a diagnostic test kit to detect human tumor antigens.

7.1. Cancer specific MAb^{p} and Cytotoxic Agents

In one embodiment, the invention provides a method for the specific destruction of cells (i.e., the destruction of tumor cells) by administering the plant-derived antibody of the invention in association with toxins or cytotoxic prodrugs.

Toxin refers to compounds that bind and activate endogenous cytotoxic effector systems, radioisotopes, holotoxins, modified toxins, catalytic subunits of toxins, or any molecules or enzymes not normally present in or on the surface of a cell that define conditions that cause the cell's death. Toxins that may be used according to the methods of the invention include, but are not limited to, radioisotopes known in the art, compounds such as, for example, antibodies (or complement fixing containing portions thereof) that bind an inherent or induced endogenous cytotoxic effector system, thymidine kinase, endonuclease, RNAse, alpha toxin, ricin, abrin, Pseudomonas exotoxin A, diphtheria toxin, saporin, momordin, gelonin, pokeweed antiviral protein, alpha-sarcin and cholera toxin.

Cytotoxic prodrug refers to a non-toxic compound that is converted by an enzyme, normally present in the cell, into a cytotoxic compound. Cytotoxic prodrugs that may be used according to the methods of the invention include, but are not limited to, glutamyl derivatives of benzoic acid mustard alkylating agent, phosphate derivatives of etoposide or mitomycin C, cytosine arabinoside, daunorubisin, and phenoxyacetamide derivatives of doxorubicin.

8. Test Kits

Also encompassed within the scope of the invention are diagnostic test kits that contain the plant-derived antibody of the invention. The antibodies are utilized in immunodiagnostic assays and kits in detectably labeled form (i.e., enzymes, fluorescent labels, etc.), or in immobilized form (on polymeric tubes, beads, etc.) They may also be utilized in labeled form for in vivo imaging, wherein the label can be a radioactive emitter, or a nuclear magnetic resonance contrasting agent such as a heavy metal nucleus, or a X-ray contrasting agent, such as a heavy metal. The antibodies can also be used for in vitro localization of the recognized tumor cell antigen by appropriate labeling.

Detection can be facilitated by coupling the antibody to a detectable agents. Examples of detectable substances include, but are not limited, to various enzymes, prosthetic groups, fluorescent materials, luminescent materials, bioluminescent materials, radioactive materials, disperse dyes, and gold particles. Examples of suitable detectable agents, as disclosed above, includes suitable enzymes, i.e., horseradish peroxidase, alkaline phosphatase, betagalactosidase, or acetylcholinesterase; examples of suitable prosthetic group complexes include, but are not limited to, streptavidin/biotin and avidin/biotin; examples of suitable fluorescent materials include, but are not limited to, umbelliferone, fluorescein, fluorescein isothiocyanate, rhodamine, dichlorotriazinylamine fluorescein, dansyl chloride or phycoerythrin; an example of a luminescent material includes, but is not limited to, luminol; examples of bioluminescent materials include, but are not limited to luciferase, luciferin, and aequorin; and examples of suitable radioactive material include, but are not limited to ¹²⁵I, ³⁵S, ¹⁴C, ³H, Tc^{99m}, or Mg⁵².

9. Pharmaceutical Compositions

The present invention also provides pharmaceutical compositions for cancer immunotherapy comprising a therapeutically effective amount of one or more plant-derived antibody of the invention or an active fragment thereof. Administration of the pharmaceutical compositions of the invention results in a detectable change in the physiology of a recipient subject, preferably by enhancing passive immunity to one or more human tumor antigens. For example, a pharmaceutical composition containing a monovalent, divalent or multivalent antibody of the present invention provides a means for treating, or ameliorating human cancers.

The pharmaceutical preparations of the present invention, are for example, in the form of sterile aqueous or non-aqueous solutions, suspensions, or emulsions, and can also contain auxiliary agents or excipients that are known in the art. A typical regimen for preventing, suppressing, or treating a disease or condition which can be alleviated by the pharmaceutical composition of the invention comprises administration of an effective amount of the composition as described above, administered as a single treatment, or repeated dosages, over a period up to and including one week to about 48 months.

According to the present invention, an “effective amount” of a composition is an amount sufficient to achieve passive immunity against cancer antigens. It is understood that the effective dosage will be dependent upon the age, sex, health, and weight of the recipient, kind of concurrent treatment, if any, frequency of treatment, and the nature of the effect desired. The most preferred dosage will be tailored to the individual subject, as is understood and determinable by one of skill in the art, without undue experimentation.

This invention is further illustrated by the following examples, which are not to be construed in any way as imposing limitations upon the scope thereof.

### EXAMPLES

### Example 1

### Construction of Plant Expression Binary Vector

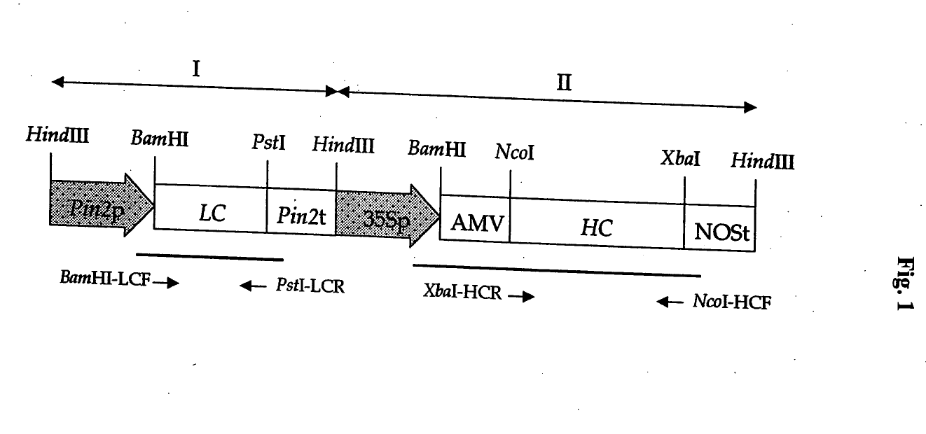

Construction of plant expression binary vector. cDNA for the HC and LC of MAb CO17-1A provided by Dr. Peter Curtis, Thomas Jefferson University, Philadelphia, Pa. was cloned into pGEM®-T vector (Verch et al. (1998) supra). The HC gene was PCR-cloned under the control of the cauliflower mosaic virus (CaMV) 35S promoter with duplicated upstream B domains and the untranslated leader sequence of alfalfa mosaic virus RNA4 in pBI525 (Datla et al. (1993) Plant Sci. 94:1398) to produce pBI525HC (FIG. 1).

To create restriction sites for cloning, forward and reverse primers were designed to contain NcoI and XbaI restriction sites on the 5′ and 3′ end of the HC gene (NcoI-HCF: 5′-cgg cca tgg aat gga gca gag tct tt-3′ (SEQ ID No: 6) and XbaI-HCR: 5′-cgt cta gat tag tga tgg tga tgg tga tga tc-3′) (SEQ ID No: 7). The LC gene was PCR-cloned under the control of Pint promoter. To clone the LC gene under the control of Pin2 promoter, the fragment of the expression cassette, Pin2p/attacinE/Pin2t, on pLDB15 (Norelli et al. (1994) Euphytica 77:123-128) was inserted into the HindIII restriction site on pGEM®-T vector. The LC gene was PCR-cloned under the control of the Pin2 promoter in pGEMT vector (Promega, Madison, Wis.) after removing the attacinE gene to yield pGEMPinLC (FIG. 1). PCR was conducted to create BamHI and PstI restriction sites using forward and reverse primers BamHI-LCF: 5′-cgg gat cca tgg gca tca aga tgg aat cac ag-3′ (SEQ ID No: 8) and PstI-LCR: 5′-cgc tgc agc taa cac tca ttc ctg ttg aag ct-3′) (SEQ ID No: 5). Expression cassettes were cloned into the plant expression binary vector pBI121 to yield pBICO17 (FIG. 1). The sequences of PCR cloned HC and LC were confirmed following standard procedures (Sanger et al. (1977) Proc. Natl. Acad. Sci. USA 74:5463-5467) using an ABI Prism 377 DNA analyzer (Applied Biosystems, Foster City, Calif.). All DNA cloning and cell transformation was performed according to standard procedures (Sambrook et al. (1989) Molecular Cloning, 2^{nd} ed. Cold Spring Harbor Laboratory Press, New York, N.Y.).

Based upon PCR and restriction endonuclease analyses, the HC and LC genes of MAb CO17-1A were confirmed to be present in pBICO17, respectively (FIG. 1). Based upon nucleotide sequencing analysis, the HC and LC genes on pBICO17 had the same as the sequences of the HC and LC genes on pGEM-T as described in previous report, respectively (Verch et al. (1998) supra). The pBICO017 vector contained the nptII gene within the T-DNA without the gus gene. To avoid the risk of transcriptional gene silencing due to the homologous gene sequence on the promoter, two different promoters, 35S and Pint promoters, were used for expression of the HC and LC genes in one plant binary vector, respectively.

### Example 2

### Plant Transformation

The plant expression binary vector pBICO17 was transferred to A. tumefaciens LBA4404 by electroporation for Agrobacterium mediated transformation. Tobacco (Nicotiana tabacum cv. Xanthi) leaf pieces were transformed according to the method of Horsh et al. (1985) supra with minor modifications. At 2 weeks after transformation the leaf pieces were transferred to MS medium containing BAP (1 μg/ml), NAA (0.1 μg/ml), carbenicillin (500 μg/ml), and kanamycin (100 μg/ml). Regenerated shoots were subcultured to MS medium containing carbenicillin (500 μg/ml) and kanamycin (100 μg/ml) to induce root. Transgenic tobacco lines were rooted, acclimated in vitro, and grown in soil pots.

Four regenerants were obtained on the regeneration media using Agrobacterium-mediated transformation. Among the four regenerants, only three regenerants had rooting on MS rooting media containing kanamycin. PCR test confirmed that the three regenerants contained the HC and LC genes and named as transgenic line T1, T2, and 24 T3, respectively. These results indicated that the regenerants not inducing root was an escape as described by McHughen and Jordan (McHughen et al. (1989) Plant Cell Rep. 7:611-164), incorporated herein by reference. There were no morphological difference between transgenic and non-transgenic plants.

### Example 3

### PCR Confirmation of Transformants

Transgenic character of plants regenerated after Agrobacterium-mediated transformation was confirmed by PCR. Regenerants were maintained in MS medium containing carbenicillin (500 μg/ml) and kanamycin (100 μg/ml). At 3 to 4 weeks in the MS medium, the genomic DNA was isolated from leaf tissues of transformed and non-transformed (control) rooted shoots by DNAeasy® Plant Mini Kit (Qiagen Inc., Valencia, Calif.). A programmable thermal controller Mastercycler® gradient (Eppendorf Scientific Inc., Germany) was used for PCR to investigate the presence of the HC and LC in regenerants. Each PCR reaction was carried out in 25 μl containing 2.5 μl of 200 μM dNTP, 1.5 mM magnesium chloride, 5 mM each primer (HC: NcoI-HCF and XbaI-HCR; LC: BamHI-LCF and Pstl-LCR), 50 ng of, genomic DNA, and 1.25 units of Taq DNA polymerase. DNA was amplified for 35 cycles of 1 min at 94° C., 1 min 55° C. and 1 min at 72° C. The amplified DNA was stained with ethidium bromide after electrophoresis on a 1% agarose gel in Tris-borate buffer (45 mM Tris-borate and 1 mM EDTA), detected at the UV light, and photographed.

Transgenic character of plants regenerated after Agrobacterium-mediated transformation was confirmed by PCR. Regenerants were maintained in MS medium containing carbenicillin (500 μg/ml) and kanamycin (100 μg/ml). At 3 to 4 weeks in the MS medium, the genomic DNA was isolated from leaf tissues of transformed and non-transformed (control) rooted shoots by DNAeasy® Plant Mini Kit (Qiagen Inc., Valencia, Calif.). A programmable thermal controller Mastercycler® gradient (Eppendorf Scientific Inc., Germany) was used for PCR to investigate the presence of the HC and LC in regenerants. Each PCR reaction was carried out in 25 μl containing 2.5 μg of 200 μM DNTP, 1.5 mM magnesium chloride, 5 mM each primer (HC: NcoI-HCF and XbaI-HCR; LC: BamHI-LCF and Pstl-LCR), 50 ng of, genomic DNA, and 1.25 units of Taq DNA polymerase. DNA was amplified for 35 cycles of 1 min at 94° C., 1 min 55° C. and 1 min at 72° C. The amplified DNA was stained with ethidium bromide after electrophoresis on a 1% agarose gel in Tris-borate buffer (45 mM Tris-borate and 1 mM EDTA), detected at the UV light, and photographed.

### Example 4

### Expression of the HC and LC of MAB CO17-1A

Western blot. Western blot analysis was conducted to confirm the expression of HC and LC in transgenic lines as described by Ko et al. (1999) Biotechnol. Tech. 13:849-857, incorporated herein by reference in its entirety. To investigate the effect of wounding on activity of the Pin2 promoter, leaves were harvested from in vivo tobacco shoot before wounding, and 1, 24, and 48 h after wounding and stored at −80° C. Leaves from tissue cultured plants were crushed with forceps. Leaf tissues of transgenic and non-transgenic tobacco were homogenized in extraction buffer (1 protease inhibitor cocktail tablet (Roche, Germany) 10 ml of 50 mM Tris, pH 7.5 and 0.2 mg of leaf fresh weight/μl). Ten μl of the extract buffer containing leaf extract was mixed with 10 μl of loading buffer and loaded onto 12% SDS-PAGE, and the proteins were then transferred to Immobilon™-P Transfer Membrane (Millipore Corp., Bedford, Mass.) using a mini-Protean II™ system (Bio-Rad Labs, CA) according to manufacturer's recommendations.

The membrane was incubated in blocking solution (0.5% (w/v) I-block™ (TROPIX, Bedford, Mass.)) in PBS plus 0.1% (v/v) Tween 20 (PBST) at 4° C. overnight with gentle agitation. The membrane was incubated in goat anti-mouse monoclonal antibody conjugated to horseradish peroxidase (catalog #115-035-062) (Jackson ImmunoResearch Labs Inc., West Grove, Pa.) in antibody solution containing 0.1% (w/v) I-block™ in PBST at room temperature for 1 and a half hour with gentle agitation. The membranes were rinsed 3 times for 10 min in PBST at room temperature. Protein bands were detected on CL-X Posure™ film (Pierce, Rockford, Ill.) using a SuperSignal® chemiluminescence substrate (Pierce, Rockford, Ill.). The MAb CO17-1A obtained from the hybridoma cell was used as a positive control according to Herlyn et al. (1986) Hybridoma 5:S3-S10, incorporated herein by reference.

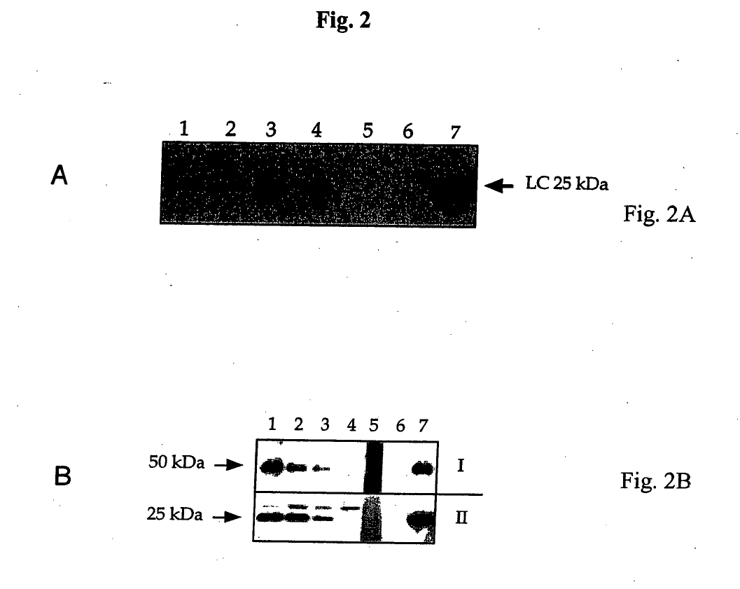

Western blot was conducted to test whether the expression of LC is wound inducible under the control of Pin2 promoter in tobacco, (FIG. 2A). With T1 transgenic tobacco line maintained in a soil pot, LC band (25 kDa) was detected before and after mechanical wounding, while non-transgenic line before or after wounding had no LC band (25 kDa), indicating that the LC gene expression was constitutive under the control of Pin2 promoter. All transgenic lines (T1, T2, and T3) had HC (50 kDa) and LC (25 kDa) protein bands (FIG. 2B). T1 had a greater density of HC and LC compared to T2 and T3 transgenic lines. The density of the HC band was positively correlated with the LC protein. Non-transgenic line had no LC or HC band. The constitutive gene expression under the control of Pin2 promoter was not wound inducible as previously reported by Sanchez-Senrano et al (1987) EMBO J. 6:303-306; and Pena-Cortós et al. (1988) Planta 174-84-89). These results were consistent with the findings of Thornburg et al. (1987) Proc. Natl. Acad. Sci. USA 84:744-748; Keinonen-Mettala et al (1998) Plant Cell Rep. 17:356-361) reporting constitutive gene expression under the control of the Pin2 promoter was observed with the GUS gene in transgenic tobacco. The results of western blot analysis demonstrated that the HC and LC proteins were produced in transgenic plant under the control of two different promoters, 35S and Pin2, respectively. These results suggest that the Pin2 promoter is an adequate promoter for LC gene expression in combination with the 35S promoter for the HC gene in a transgenic plant.

### Example 5

### Binding Activity of an Assembled Full-Size MAB CO17-1A for GA733-2E

To determine whether the HC and LC proteins of MAb CO17-1A are assembled and functional to bind Ag GA733-2, leaf extracts of T1, T2, and T3 transgenic lines and non-transgenic line were applied to ELISA plates coated with the Ag GA733-2E.

ELISA plates, 96-well Nunc-Immuno™ MaxiSorp™ Surface plates (Nunc, Denmark) were coated with 1 μg/ml of the Ag GA733-2E (Strassburg et al. (1992) Cancer Res. 52:815-821, incorporated herein by reference), in 50 mM sodium carbonate at pH 9.6 for 1 h at 37° C. Leaf tissues were harvested from the transgenic and non-transgenic tobacco lines maintained in soil. Protein was extracted by grinding 20 mg of young leaf tissue in 100 μl of an extraction buffer that consisted of 10 mM sodium sulfite, 2% (w/v) polyvinylpyrrolidone (MW 40,000), 3 mM sodium azide, and 2% (v/v) Tween-20. The plates were loaded with 50 μl of tobacco plant extracts of transgenic and non-transgenic lines and 50 μl of serial threefold dilutions of 2 μg/ml of MAb CO17-1A purified from the hybridoma supernatant (Herlyn et al. (1986) supra) as a positive control, and incubated overnight at 4° C. After washing the plate 3 times with 1×PBS and 0.05% (v/v) Tween-20, horseradish peroxidase conjugated goat anti-mouse antibody (catalog #115-035-062) (Jackson ImmunoResearch Labs, INC., West Grove, Pa.) was loaded and incubated 1 h at room temperature. After washing the plate 5 times, 50 μl of o-phenylenediamine dihydrochloride prepared based upon the manufacturer's recommendation (Sigma, St. Louis, Mo.) were loaded for a peroxidase substrate. The experiment was performed with two leaf samplings of each line. The absorbance was read using a SPECTRAmax® 340PC Microplate Spectrophotometer (Molecular Devices, Sunnyvale, Calif.).

Cell ELISA. 100 μl of Ag GA733-2 expressing colorectal carcinoma cell line SW948 and negative control melanoma cell line WM115 (1×10⁶ cell/ml) were added into B-D Falcon 96-well assay flat-bottom plates (Becton Dickinson, Franklin Lakes, N.J.). After overnight incubation at 37° C., the media solution was discarded. The cells in plates were fixed in 50 μl of 0.05% glutaraldehyde in 1×PBS for 20 min at room temperature. The plates were washed four times with 1×PBS and blocked with 25 μl of 0.7% glycine. Sample preparation of transgenic and non-transgenic tobacco lines, and ELISA procedures were conducted as described in Materials and Methods. 0.93 μg/ml of MAb CO17-1A purified from hybridoma supernatant was included to this assay as a control. Statistical significance of immunological data was calculated with Student's t test using MINITAB™ statistical software (Minitab Inc., State College, Pa.).

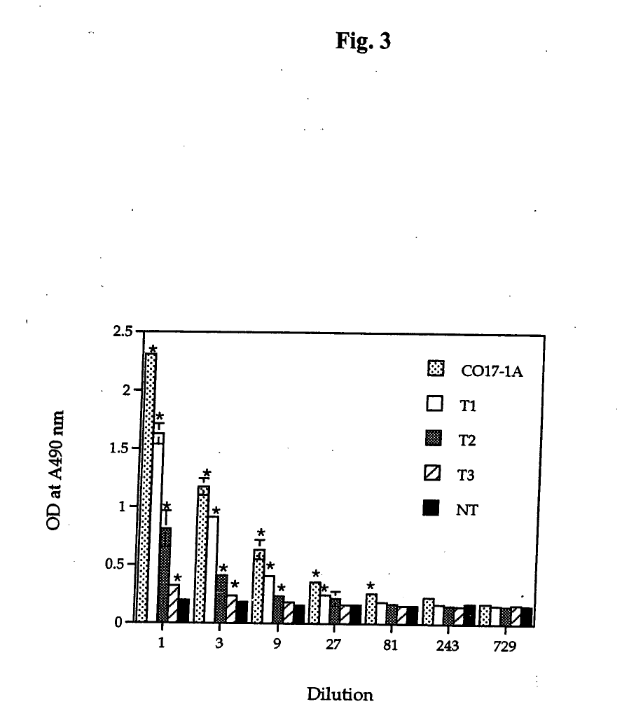

The results indicated that all transgenic lines had significantly greater absorbance value (OD at 490 nm) than non-transgenic line with a background signal (p<0.05 at dilution 1:1 and 1:3) (FIG. 3). Among three transgenic lines, T1 with the highest density of HC and LC bands had the significantly greater value up to 1:27 while T3 with the lowest density of HC and LC bands had the significantly greater value up to 1:3. The concentration MAb CO17-1A in T1 plant extracts was 0.93 μg/ml T1 with the highest expression of both HC and LC (FIG. 2B) had the expression level 0.073 to 1% of total soluble protein of leaf. The expression level was similar to a previous report that the murine IgG expressed in tobacco leaf ranged from 0.05 to 0.4% (see, for example, van Engelen et al. (1994) Plant Mol. Biol. 26:1701-1710). The ELISA results indicated that the HC and LC proteins produced in tobacco are assembled and functional to bind the Ag GA733-2E.

The Ag GA733-2E is a recombinant antigen produced from the baculovirus-insect cell expression system with the Ag GA733-2E gene, which is truncated of transmembrane and cytoplasmic domains (Strassburg et al.(1992) supra). Therefore, the recombinant Ag GA733-2E might be differently folded compared to the native antigen GA733-2 on colorectal carcinoma cells, resulting in changed immunoreactivity to antibody (Akis et al (2002) J. Immunol. Meth. 261:119-127). To further confirm whether the MAb CO17-1A produced in transgenic tobacco has specific binding activity to the native Ag GA733-2, the leaf extract was applied to the ELISA plate coated with colorectal carcinoma cell line SW948 expressing the native Ag GA733-2 and a negative control cell line WM115 which lacks the GA733-2 antigen. The purified MAb 1 CO17-1A (0.93 μg/ml) from hybridoma (I and II) and leaf extract of T1 plant producing MAb CO17-1A gave significantly higher absorbance (0.933, 0.896, and 0.807) in SW 948 than WM115 (0.111, 0.103, and 0.113), respectively (p<0.05) (Table 1). Leaf extract of non-transgenic plant had background absorbance values (0.115 and 0.108) in both cell lines SW948 and WM115, respectively. The purified MAb CO17-1A (0.93 μg/ml) from hybridoma (I and II) and leaf extract of T1 plant producing MAb CO17-1A were not significantly different (p<0.05) (Table 1). These results indicated that plant leaf extracts do not hinder the binding activity of MAb CO17-1A, and the MAb CO17-1A produced in transgenic tobacco specifically binds the colorectal carcinoma SW948 similar to MAb CO17-1A from hybridoma.

The present invention may be embodied in other specific methods, products, and forms without departing from its spirit of essential characteristics. The embodiments and examples provided in this specification are intended to illustrate the principles of the invention, but not to limit its scope. Various other embodiments, examples, modifications, and equivalents to the embodiments and examples provided in this specification may occur to those skilled in the art upon reading the present disclosure or practicing the present invention. Such variations, modifications, examples, and equivalents are intended to come within the scope of the invention. The contents of all references, patents and published patent applications cited throughout this application are expressly incorporated herein by reference.

1. An expression vector comprising one or more gene constructs comprising polynucleotides encoding CO17-1A MAb^{p} under the control of one or more promoters, operatively linked to one or more regulatory control elements and Agrobacterium T-DNA terminal repeats.

2. The expression vector of claim 1, wherein the one or more regulatory control elements comprise an alfalfa mosaic virus untranslated leader sequence.

3. The expression vector of claim 1, wherein the CO17-1A MAb^{p} comprises one or more CO17-1A MAb^{p} subunits.

4. The expression vector of claim 3, wherein the one or more CO17-1A MAb^{p} subunits comprise CO17-1A MAb heavy chain, light chain, or both, and the alfalfa mosaic virus untranslated leader sequence is linked at the N-terminus of the heavy chain.

5. The expression vector of claim 4, wherein the heavy chain, the light chain or both are under control of one or more promoters.

6. The expression vector of claim 5, wherein the one or more promoters comprise one or more constitutive promoters.

7. The expression vector of claim 6, wherein the constitutive promoters comprise a cauliflower mosaic virus 35S promoter with duplicated upstream B domains, and a potato proteinase inhibitor II promoter.

8. The expression vector of claim 1, wherein the one or more gene constructs further comprise nucleic acid sequences encoding an endoplasmic reticulum retention signal.

9. The expression vector of claim 8, wherein the endoplasmic reticulum retention signal is SEQ ID NO: 9 or SEQ ID NO: 10.

10. The expression vector of claim 1, wherein the expression vector is pBICO17.

11. A transgenic plant comprising the expression vector of claim 1.

12. The transgenic plant of claim 11, wherein the expression vector is pBICO17.

13. The transgenic plant of claim 11, wherein the transgenic plant is a tobacco plant.

14. The transgenic plant of claim 13, wherein the tobacco plant comprises a whole plant, plant cells, tissues, and organs of the tobacco plant.

15. A plant-derived human monoclonal antibody comprising a CO17-1A MAb^{p}, wherein the CO17-1A MAb^{p} contains predominantly oligomannose type N-glycans and has substantially reduced or no α(1,3)-linked fucose residues.

16. A plant-derived human monoclonal antibody comprising a CO17-1A MAb^{p}, wherein the CO17-1A MAb^{p} comprises an endoplasmic reticulum retention signal, and contains about 70% oligomannose type N-glycans.

17. The plant-derived human monoclonal antibody of claim 16, wherein the antibody contains about 70% Man₆₋₉GlcNAc₂, about 3% GlcNAc₂Man₃GlcNAc₂, and about 3% GlcNAc₂(Xyl)Man₃GlcNAc₂.


SEQUENCE LISTING (11 sequences)

SEQ ID NO: 1 - DNA, 1389 bp, Mus musculus
atggaatggagcagagtctttatctttctcctatcagtaactgcaggtgttcactcccaggtccagttggtcgactctggagctgagctggtaaggcctgggacttcagtgaaggtgtcctgcaaggcttctggatacgccttcactaattacttgatagagtgggtaaagcagaggcctggacagggccttgagtggattggggtgattaatcctggaagtggtggtactaactacaatgagaagttcaagggcaaggcaacactgactgcaggcaaatcctccagcactgcctacatgcagctcagcagcctgacatctgatgactctgcggtctatttctgtgcaagagatggtccctggtttgcttactggggccaagggactctggtcactgctctgcaggccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggacttaa

SEQ ID NO: 2 - protein, 463 aa, Mus musculus
MEWSRVFIFLLSVTAGVHSQVQLVDSGAELVRPGTSVKVSCKASGYAFTNYLIEWVKQRPGQGLEWIGVINPGSGGTNYNEKFKGKATLTAGKSSSTAYMQLSSLTSDDSAVYFCARDGPWFAYWGQGTLVTALQAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTZ

SEQ ID NO: 3 - DNA, 717 bp, Mus musculus
atgggcatcaagatggaatcacagactctggtcttcatatccatactgctctggttatatggagctgatgggaacattgtaatgacccaatctcccaaatccatgtccatgtcagtaggagagagggtcaccttgacctgcaaggccagtgaaaatgtggttacttatgtttcctggtatcaacagaaaccagagcagtctcctaaactgctgatatacggggcatccaaccggtacactggggtccccgatcgcttcacaggctcaggatctgcaacagatttcactctgaccatcagcagtgtgcaggctgaagaccttgcagattatcactgtggacagggttacagctatccgtacacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 4 - protein, 239 aa, Mus musculus
MGIKMESQTLVFISILLWLYGADGNIVMTQSPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECZ

SEQ ID NO: 5 - DNA, 32 bp, Artificial Sequence
  Primer for MAb CO17-1A light chain DNA
cgctgcagctaacactcattcctgttgaagct

SEQ ID NO: 6 - DNA, 26 bp, Artificial Sequence
  Primer for MAb CO17-1A heavy chain DNA
cggccatggaatggagcagagtcttt

SEQ ID NO: 7 - DNA, 32 bp, Artificial Sequence
  Primer for MAb CO17-1A heavy chain DNA
cgtctagattagtgatggtgatggtgatgatc

SEQ ID NO: 8 - DNA, 32 bp, Artificial Sequence
  Primer for MAb CO17-1A light chain DNA
cgggatccatgggcatcaagatggaatcacag

SEQ ID NO: 9 - protein, 4 aa, Artificial Sequence
  ER retention signal
KDED

SEQ ID NO: 10 - protein, 4 aa, Artificial Sequence
  ER retention signal
HDED

SEQ ID NO: 11 - DNA, 12 bp, Artificial Sequence
  ER retention coding sequence
gagctcatcttt